(19) United States
(12) Patent Application Publication
(10) Pub. No.: US 2019/0192033 A1
(43) Pub. Date: Jun. 27, 2019

(54) NEUROFEEDBACK SYSTEMS AND METHODS

(71) Applicant: Paul NURFLUS, Tzur-Yigal (IL)

(72) Inventors: Paul NURFLUS, Tzur-Yigal (IL); Einat NURFLUS, Tzur-Yigal (IL)

(73) Assignee: BestBrian Ltd., Tzur-Yigal (IL)

(21) Appl. No.: 16/098,894

(22) PCT Filed: May 4, 2017
(86) PCT No.: PCT/IL2017/050496

§ 371 Date: Nov. 5, 2018

Related U.S. Application Data
(60) Provisional application No. 62/332,089, filed on May 5, 2016.

(51) Int. Cl. A61B 5/0482; A61B 5/00

(52) U.S. Cl. CPC A61B 5/0482; A61B 5/7246; A61B 5/7207; A61B 5/7267; A61B 5/4836; A61B 5/6803; A61B 5/4064

(57) ABSTRACT

There is provided a computer implemented method for adapting a neurofeedback treatment, comprising: receiving at least one patient brain state parameter indicative of a current brain state of a patient for application of a neurofeedback treatment; correlating the at least one patient brain state parameter with a set of neurofeedback treatments from a plurality of neurofeedback treatments stored in a dataset; iterating for members of the set of neurofeedback treatments: selecting one neurofeedback treatment from the set of neurofeedback treatments, wherein in each iteration another neurofeedback treatment is selected; administering the one neurofeedback treatment to the patient; calculate an effectiveness parameter associated with the one neurofeedback treatment administered to the patient based on measured outputs of at least one brain signal outputted by at least one sensor sensing the head of the patient; and designating an effective neurofeedback treatment according to the measured effectiveness parameter.

### BACKGROUND

The present invention, in some embodiments thereof, relates to neurofeedback and, more specifically, but not exclusively, to systems and methods for administering neurofeedback treatments.

Neurofeedback is a type of biofeedback that measures brain waves of a patient to produce a signal that is used as feedback back to the brain of the patient, teaching the brain of the patient to self-regulate. Neurofeedback may cause neuroplastic changes after treatments, for example, an increase in brain size.

Neurofeedback is commonly provided using video or sound. A positive feedback, such as good video contrast and audio quality is used to encourage the desired brain activity. Negative feedback, such as poor contrast or reduced audio quality is used to discourage brain activity that is undesirable. In this manner, the brain of the patient watching the video or hearing the sound learns to create the designed brain waves by being rewarded with the ability to watch the video or hear the audio with good quality.

Despite being shown as effective for treating a variety of brain based disorders, Neurofeedback is currently not readily available to many patients. Neurofeedback requires about 30 to 40 treatment sessions to be durably effective. Each session is administered by an experienced Neurofeedback treatment doctor that determines the treatment, monitors progress, and helps ensures success. As such, Neurofeedback may only be available at dedicated clinics which are not always easily reachable. Neurofeedback has a relatively high cost of treatment.

The neurofeedback therapy selected for each patient is highly dependent on the therapist. The therapist selects the therapy, for example, based on acquired neurofeedback knowledge, personal experience, diagnosis capability, associative thinking, and ability to analyze other patient related data. Neurofeedback treatments selected by therapists are estimated as having an average success rate of about 70-80% depending on the pathology treated.

### SUMMARY

According to an aspect of some embodiments of the present invention there is provided a computer implemented method for adapting a neurofeedback treatment, comprising: receiving at least one patient brain state parameter indicative of a current brain state of a patient for application of a neurofeedback treatment; correlating the at least one patient brain state parameter with a set of neurofeedback treatments from a plurality of neurofeedback treatments stored in a dataset; iterating for members of the set of neurofeedback treatments: selecting one neurofeedback treatment from the set of neurofeedback treatments, wherein in each iteration another neurofeedback treatment is selected; administering the one neurofeedback treatment to the patient; calculate an effectiveness parameter associated with the one neurofeedback treatment administered to the patient based on measured outputs of at least one brain signal outputted by at least one sensor sensing the head of the patient; and designating an effective neurofeedback treatment according to the measured effectiveness parameter.

Optionally, the effectiveness parameter is calculated as the sum of a time duration of each respective appearance event of a value calculated for a target signal pattern determined based on output of at least one brain signal. Optionally, the time duration of each respective appearance event of the value is determined based on a threshold requirement represent a local maximum of the target signal pattern or a local minimum of the target signal pattern. Optionally, the value calculated for the target signal pattern determined based on output of at least one brain signal comprises a power value of each appearance event of a target type of brain activity calculated from electroencephalogram (EEG) signals.

Optionally, the effectiveness parameter is calculated regardless of whether a reward threshold of the administered neurofeedback treatment is met or not.

Optionally, the method further comprises administering the selected effective neurofeedback treatment to the patient for a predefined range of time longer than the range of time of administration of each respective neurofeedback treatment.

Optionally, the iterating is performed for a subset of neurofeedback treatments, the selected effective neurofeedback treatment is selected and administered, and another iterating is performed for the remaining members of the set of neurofeedback treatments that were not members of the iterated subset. Optionally, the method further comprises repeating the iterating and the selecting to select another effective neurofeedback treatment, and administering the other selected effective neurofeedback treatment to the patient for another predefined range of time longer than the predefined range of time of administration of the previous effective neurofeedback treatment.

Optionally, the method further comprises associating each member of the set of neurofeedback treatments with a plurality of treatment parameters each representing a different value for a requirement target, wherein a calculation based on output of at least one sensor measuring brain signals of the patient is compared to the value of the requirement target. Optionally, at least one of an image and a sound is modulated according to the comparison of the calculation based on the output of the at least one sensor to the value of the requirement target. Optionally, selecting comprises selecting one neurofeedback treatment from the set of neurofeedback treatments and an associated set of treatment parameters selected from the plurality of treatment parameters, and measuring the effectiveness parameter according to the associated set of treatment parameters. Optionally, iterating comprises iterating the combination of neurofeedback treatments and the plurality of treatment parameters.

Optionally, during each iterating, each one neurofeedback treatment is administered for an approximately equal range of time.

Optionally, the method further comprises: administering an evaluation to obtain a first score for the patient at the current brain state of a patient, administering the evaluation to obtain a second score for the patient after the effective neurofeedback treatment is selected, and comparing the first and second scores.

Optionally, the method further comprises removing the effective neurofeedback treatment from use in the iterating when the comparison of the first and second scores is not statistically significant.

Optionally, the at least one patient brain state parameter is selected from the group consisting of: memory improvement, attention improvement.

Optionally, when the at least one patient brain state parameter comprises memory improvement, the set of neurofeedback treatments comprise at least one of: absolute power value of the Alpha frequency measured at a selected electrode, relative power of the Alpha frequency compared to the power of the rest of all other frequencies measured for the selected electrode, average power of the measured Alpha frequency over time, and coherence between the phases of the Alpha frequency of several electrodes.

Optionally, each one neurofeedback treatment is randomly from the set of neurofeedback treatments without repeating selection of a previously selected neurofeedback treatment.

According to an aspect of some embodiments of the present invention there is provided a computer implemented method for creating a placebo of a neurofeedback treatment, comprising: administering a non-therapeutic neurofeedback treatment to a patient that simulates a neurofeedback treatment without reacting to brain signals according to a real neurofeedback treatment; detecting an artifact in output of at least one sensor measuring at least one signal; and presenting output in response to the detected artifact.

Optionally, the artifact is based on measurements in response to patient activity unrelated to the administration of the non-therapeutic neurofeedback.

Optionally, the artifact represents at least one signal generated in response to a member selected from the group consisting of: a blink of the eye of the patient, movement of the at least one sensor, movement of the head of the patient, chewing movement by the patient, and movement of the limb or body of the patient.

Optionally, the non-therapeutic neurofeedback treatment comprises randomly adapting at least one of an image and a sound in a manner similar to the real neurofeedback treatment that is un-correlated with a measured at least one brain signal of the patient.

Optionally, the artifact is detected in EEG signals measured based on output of the at least one sensor.

Optionally, the artifact comprises a power spike indicative of saturation in the output of the at least one sensor.

Optionally, the artifact is detected based on an analysis of non-EEG signals measured based on output of the at least one sensor.

Optionally, presenting output in response to the detected artifact comprises presenting a simulation of output that is presented in response to the real neurofeedback treatment using at least one EEG sensor measuring at least one EEG signal of the patient.

According to an aspect of some embodiments of the present invention there is provided an element for placement of a neurofeedback headset at a predefined position on a head of a patient, comprising: a first end portion for coupling to an anterior portion of the neurofeedback headset; an elongated portion extending from the first end portion, the elongated portion having a length such that when the neurofeedback headset is located at the predefined positioned the elongated portion extends parallel to the surface of the frontal bone of the patient until the glabella of the patient; a pair of arms each extending laterally in opposite directions and inferiorly from the end portion of the elongated portion positioned at the glabella, each respective arm oriented for positioning along at least one of the respective side of the nasal bone and inferiorly to the respective eyebrow and superiorly to the respective eye of the patient.

Optionally, the elongated portion is positioned and biased to contact the skin surface of the frontal bone.

Optionally, the length of the elongated portion is adjustable to fit patients having different fontal bone surface sizes.

Optionally, the end portion of each arm of the pair of arms includes a contact element for contacting the skin of the patient, wherein the contact element is sized and positioned away from the respective eye of the patient.

Optionally, the end portion of each arm of the pair of arms is positioned medially to a respective supraorbital notch.

Optionally, when the neurofeedback headset comprises a plurality of EEG electrodes that output EEG signals when contacting the head of the patient when the neurofeedback headset is positioned at the predefined position.

According to an aspect of some embodiments of the present invention there is provided a system for testing a neurofeedback headset, the system comprising: a cradle comprising: a plurality of electrical elements for outputting at least one signal; a surface shaped to accommodate an inner surface of the neurofeedback headset that contacts a head of a patient when the patient wears the neurofeedback headset, the surface shaped according to a size and shape of the head of the patient; wherein the plurality of electrical elements are arranged along the surface of the cradle such that when the neurofeedback headset is located on the surface of the cradle, electrodes located within the neurofeedback headset are in electrical communication with the plurality of electrical elements of the surface; and a controller that controls the plurality of electrical elements to create at least one test signal for reception by the electrodes of the neurofeedback headset.

Optionally, the plurality of electrical elements are transmitters designed to create electrical communication by contacting the electrodes of the neurofeedback headset when the neurofeedback headset is positioned on the surface of the cradle.

Optionally, the plurality of electrical elements are electromagnetic generators designed to create wireless electrical communication with the electrodes of the neurofeedback headset by non-contact with the electrodes when the neurofeedback headset is positioned on the surface of the cradle.

Optionally, the controller controls the plurality of electrical elements to generate signals mimicking EEG.

Optionally, the controller controls the plurality of electrical elements to generate impedance signals representing electrical connectivity between the electrical elements and the electrodes.

Optionally, the system further comprises: a computing unit in electrical communication with the neurofeedback headset, the computing unit including a processor in communication with a program store storing code instructions for execution by the processor, the code instructions comprising: code to receive a plurality of test signals from the neurofeedback headset, wherein the plurality of test signals are generated by the controller, transmitted by the electrical elements, and sensed by the electrodes of the neurofeedback headset; and code to analyze the plurality of test signals to verify proper functionality of the neurofeedback headset.

Optionally, the analysis is performed by correlating the plurality of test signals to expected signals according to a correlation requirement representing a tolerance of proper functionality. Optionally, the system further comprises code to present a message indicative of improper functionality on a display in communication with the computing unit when the verification of proper functionality fails.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart of a method for automatically administering neurofeedback treatment to a patient, in accordance with some embodiments of the present invention;

FIG. 2 is a diagram of components of a system that automatically administers neurofeedback treatment to a patient, in accordance with some embodiments of the present invention;

FIG. 3 is a flowchart of a method for automatically verifying function of a neurofeedback headset, in accordance with some embodiments of the present invention;

FIG. 4 is a flowchart of a method for selecting and/or adapting a neurofeedback treatment for patient, in accordance with some embodiments of the present invention;

FIG. 5 is a flowchart of a method for creating a placebo neurofeedback treatment, in accordance with some embodiments of the present invention;

FIG. 6A is a schematic depicting a position element for positioning a neurofeedback headset at a predefined location on the head of a patient, in accordance with some embodiments of the present invention;

FIG. 6B is a blow-up of the position element, in accordance with some embodiments of the present invention;

FIG. 7 is a schematic of an example of a professional implementation of the system of FIG. 2, in accordance with some embodiments of the present invention;

FIG. 8 is a schematic of an example of a home implementation of the system of FIG. 2, in accordance with some embodiments of the present invention; and

FIG. 9 is a flowchart of another exemplary method of selecting effective neurofeedback treatments based on calculation of the effectiveness parameters, in accordance with some embodiments of the present invention.

### DETAILED DESCRIPTION

The present invention, in some embodiments thereof, relates to neurofeedback and, more specifically, but not exclusively, to systems and methods for administering neurofeedback treatments.

An aspect of some embodiments of the present invention relates to systems and/or methods (e.g., the method implemented by code instructions executed by a processor) that select and/or adapt a neurofeedback treatment from multiple available neurofeedback treatments to a patient. Different neurofeedback treatments associated with a patient brain state parameter (e.g., associated with a medical condition designated for improvement optionally in associated with a medical diagnosis, for example, memory secondary to onset of Alzheimer's, and concentration secondary to attention deficit disorder (ADD)) are administered to the patient. An effectiveness parameter is defined, and measured for each administered neurofeedback treatment.

The neurofeedback treatment with the highest effectiveness parameter is selected for administration to the patient. The systems and/or methods select the neurofeedback treatment that is most effective for the current patient from a set of available neurofeedback treatments that may be used to treat patients having the same or similar brain state parameter. Since the brain of each patient is unique, different neurofeedback treatments may have different levels of effectiveness for different patients.

The effectiveness parameter represents a measure of the effectiveness of the neurofeedback treatment being administered to the patient. The effectiveness parameter provides an absolute measurement of the activity of the brain of the patient, by calculating one or more values based on brain signals, independent of the actual neurofeedback treatment being administered, and/or independent of the reward threshold of the treatment being administered. The effectiveness parameter may be calculated as an integral or sum optionally of a function applied to the sensed brain signals, for example, a weighted average of the signal strength of a certain brain signal type (e.g., alpha waves) and/or of a certain frequency (e.g., 8-12 Hertz) measured by a certain electrode located on a certain portion of the brain (e.g., occipital).

The effectiveness parameter utilizes sensed values of the brain signals independently of whether a reward threshold is met or not being met according to the applied neurofeedback treatment. It is noted that the neurofeedback treatment being applied rewards the patient when a threshold is met, and does not reward the patient when a threshold is not being met. The effectiveness parameter captures the actual activity of the brain signals during the neurofeedback treatment, by incorporating into the calculation the brain signal activity when the reward threshold is not being met, and incorporating the brain signal activity when the reward threshold is being met.

The effectiveness parameter allows fine tuning of the neurofeedback treatment, for example, by providing data that may be used to adjust the reward threshold of the treatment, and/or select a different treatment that relates to different brain signals.

The effectiveness parameter captures data indicative of what the brain signal activity looks like when the reward threshold of the applied neurofeedback treatment is not being met, for example, whether the brain signal activity is close to the reward threshold, or far away from the reward threshold. In the former case, adjustment of the reward threshold to a lower level may improve effectiveness of the treatment (e.g., current neurofeedback treatment threshold too difficult for the patient). In the latter case, a different neurofeedback treatment may be selected. The effectiveness parameter captures data indicative of what the brain signal activity looks like when the reward threshold of the applied neurofeedback treatment is being met, for example, whether the brain signal activity is slightly above the reward threshold, or greatly above the threshold. In the latter case, the reward threshold may be increased (e.g., current neurofeedback treatment is too easy for the patient). The former case may represent an effective neurofeedback treatment.

The effectiveness parameter is used to select an effective neurofeedback treatment, optionally the most effective neurofeedback treatment, from a set of neurofeedback treatments, which may vary in effectiveness. The effective neurofeedback treatment may be selected based on the effectiveness parameter automatically (as described herein), which may be performed without manual intervention by a physician or other neurofeedback therapist, for example, using stand-alone equipment which may be used by the patient at home.

The effectiveness parameter is calculated based on sensed brain signals, for example, calculated based on EEG measurements, that are typically discarded by standard neurofeedback methods that simply count whether a reward threshold is met or not. Using the extended set of EEG data (i.e., not used to count rewards by standard neurofeedback methods) the actual effect of the neurofeedback treatment on the patient may be estimated. Such an evaluation of the effectiveness of the neurofeedback treatment cannot be estimated based on reward counting. For example, to determine whether a certain neurofeedback treatment is effective in strengthening the alpha waves on a certain electrode, the sum, integral, and/or weighted average of signals (e.g., all received signals, or subset of signals) may be used to calculate the effectiveness parameter.

It is noted that the systems and/or methods described herein, that calculate the effectiveness parameter, operate differently than other neurofeedback methods that are based on counting the number of rewards provided to the patient. Inventors discovered that counting rewards does not take into account, for example, the appearance(s) of desired brain wave activity of the patient when the desired activity appears below the threshold set for administering rewards (e.g., the effectiveness of the neurofeedback treatment is hidden). Moreover, counting rewards does not take into account the appearance of designed brain wave activity between threshold periods. Using the effectiveness parameter, effective neurofeedback treatments may be identified, even when such treatments appear to be ineffective using existing methods based on counting reward thresholds. The hidden effectiveness of neurofeedback treatments may be discovered using the effectiveness parameter, allowing selection of the most effective neurofeedback treatment.

The effectiveness parameter allows automatic selection of the most effective neurofeedback treatment adapted to the brain of each patient. For example, for a given medical condition (e.g. attention deficit hyperactivity disorder (ADHD)), various neurofeedback treatment types and/or threshold levels may be effective at various levels of effectiveness. The effectiveness and/or reward threshold setting of each neurofeedback treatment may be different for each patient, since each patient's brain responds differently to the same treatment and/or the same treatment reward threshold level. The same neurofeedback treatment which is effective for one patient may be ineffective for another patient diagnosed with the same medical condition. Using the systems and/or method described herein, the best (i.e., most effective) neurofeedback treatment may be automatically selected for the brain of each patient, based on the effectiveness parameter calculated for each actual neurofeedback treatment applied to the brain of each patient. The effectiveness parameter identifies the level of effectiveness of each neurofeedback treatment independently for each patient, which helps avoid situations in which a neurofeedback treatment which is ineffective for one patient but has been manually selected by a neurofeedback therapist since the treatment is found to be effective for another patient.

The systems and/or methods described herein provide a technical solution to the technical problem of automatically measuring an effectiveness of an applied neurofeedback treatment. The systems and/or methods described herein calculate an effectiveness parameter, which allows comparing one treatment to another to determine the most effective treatment, and/or comparing one treatment to an absolute measure to determine whether the neurofeedback treatment is effective or not and/or the amount of effectiveness of the neurofeedback treatment.

The systems and/or methods described herein provide a technical solution to the technical problem of automatically selecting an effective neurofeedback treatment for a patient and/or adjusting parameter of a neurofeedback treatment to improve the effectiveness of the neurofeedback treatment. The determined effective neurofeedback treatment is designed for automatic administration to the patient using the systems and/or methods described herein.

The systems and/or methods described herein tie mathematical operations (e.g., calculating the effectiveness parameter for each neurofeedback treatment) to the ability of a processor to execute code instructions, for example, by selecting different the neurofeedback treatment plans, selecting different treatment parameters, administering different neurofeedback treatment plans optionally with different treatment parameters, calculating the effectiveness parameter for each administered neurofeedback treatment, and determining the most effective neurofeedback treatment.

The systems and/or methods described herein improve performance of a system that applies neurofeedback treatments (e.g., neurofeedback headset measuring brain signals, and/or computing unit responsive to the measured brain signals that adjusts video and/or audio presented to the patient), by improving the selection of the neurofeedback treatment to administer to each patient.

Accordingly, the systems and/or methods described herein are necessarily rooted in computer technology to overcome an actual technical problem arising in the technical field of automatic administration of neurofeedback therapy.

An aspect of some embodiments of the present invention relates to systems and/or methods (e.g., the method implemented by code instructions executed by a processor) that administer a placebo of a neurofeedback treatment, for example, as part of a clinical trial evaluating a real neurofeedback treatment. The placebo simulates the real neurofeedback treatment without reacting to actual brain signals of the patient, for example, electroencephalogram (EEG). Artifacts in outputs of sensors are detected, for example, artifacts that would be detected in EEG signals during the real neurofeedback treatment. Such artifacts are unrelated to brain signals of the patient, and may be caused by patient movement, for example, blinking of eyes, chewing movement, touching the electrodes, and movement of the head. An output is presented in response to the detected artifacts. The output simulates the response that is provided when such artifact is detected in the real neurofeedback system. In this manner, by responding to patient caused artifacts, patients receiving placebo therapy cannot detect (or have a higher time detecting) whether they are receiving placebo or real neurofeedback treatment.

The systems and/or methods described herein provide a technical solution to the technical problem of simulating a placebo treatment of a neurofeedback treatment. The placebo treatment is designed to be difficult for the patient to detect using simple actions, for example, by tapping the electrode and determining whether a response is detected or not.

The systems and/or methods described herein tie mathematical operations (e.g., simulating a response to patient actions sensed by electrodes) to the ability of a processor to execute code instructions, for example, by creating output in a placebo neurofeedback treatment that simulates output that would be presented in a real neurofeedback treatment based on patient actions such as movement of eyes, jaw, or limbs.

The systems and/or methods described herein improve performance of a system that tests administered neurofeedback treatments (e.g., neurofeedback headset measuring brain signals, and/or computing unit responsive to the measured brain signals that adjusts video and/or audio presented to the patient), by providing a placebo neurofeedback treatment that is difficult for the patient to detect. The placebo neurofeedback treatment improves clinical trials, by allowing for single and/or double blind trials of neurofeedback treatments.

Accordingly, the systems and/or methods described herein are necessarily rooted in computer technology to overcome an actual technical problem arising in the technical field of testing neurofeedback therapy.

An aspect of some embodiments of the present invention relates to an element for placement of a neurofeedback headset at a predefined position on the head of the patient. The element has one end portion that couples to the neurofeedback headset, and an elongated portion that extends inferiorly along the skin surface of the frontal bone to the glabella. At the glabella, the element splits into a pair of arms, each extending inferiorly and laterally to opposite sides. The arms are shaped to contact the patient inferior to each eyebrow (and superiorly to the each eye) and/or at the respective side of the nasal bone. The element helps the patient repeatedly position the neurofeedback headset at the same (or similar) predefined position. Electrodes located within the neurofeedback headset sense brain signals (e.g., EEG) at the same (or similar) locations on the head of the patient.

The position element is shaped to feel comfortable to the patient and recognizable by contact with the skin, such that the patient easily positions the position element at similar locations at each treatment session. The position element is designed for limited displacement when located at the correct anatomical location on the face (i.e., by the elongated portion splitting into the arms that contact the skin below the eyebrows) such that movement of the neurofeedback headset away from the defined position is easily determined by the patient. The patient will feel when the neurofeedback headset moves away from the predefined position by movement of the position element across the skin of the face of the patient. The position element is not necessarily designed to secure the neurofeedback headset in position, but rather to help the patient determine when the neurofeedback headset is in the correct predefined location.

The systems and/or methods described herein provide a technical solution to the technical problem of repeatedly positioning a neurofeedback headset at the same or similar location on the head of the patient. The positioning helps ensure that electrodes located within the neurofeedback headset are positioned at the correct places on the head of the patient. The positioning allow for administration of neurofeedback treatments in sequential sessions based on brain signals measured at the same or similar locations on the head.

The systems and/or methods described herein tie mathematical operations (e.g., measuring brain signals, such as EEG, and adapting an image or audio presentation in response) to the ability of a processor to execute code instructions, for example, by allowing for repeated position of electrodes on the head of the patient, multiple neurofeedback sessions may be automatically administered correctly based on the assumptions that at each session the electrodes measure similar brain signals. The repeated positioning reduces or prevents calibration of the electrodes and/or the system.

The systems and/or methods described herein improve performance of a system that applies neurofeedback treatments (e.g., neurofeedback headset measuring brain signals, and/or computing unit responsive to the measured brain signals that adjusts video and/or audio presented to the patient), by allowing for the neurofeedback session to be administered at different treatment session without necessarily requiring calibration and/or determining the correct position of the electrodes.

Accordingly, the systems and/or methods described herein are necessarily rooted in mechanical design technology to overcome an actual technical problem arising in the technical field of mechanical headset design.

An aspect of some embodiments of the present invention relates to systems and/or methods for testing the neurofeedback headset. The system includes a cradle having a surface shaped to accommodate the inner surface of the neurofeedback headset, for example, the cradle is shaped based on the head of the patient. The cradle includes electrical elements (e.g., transmitters) that create test signals (based on instructions of an associated controller) that are sensed by the electrodes of the neurofeedback headset. The received test signals are analyzed (e.g., by a computing unit in communication with the neurofeedback headset) to determine whether the neurofeedback headset is functioning correctly. When an error is detected, a message indicative of the error is outputted, for example, presented on a display. In this manner, the neurofeedback headset may be tested before and/or after each neurofeedback treatment session.

The systems and/or methods described herein provide a technical solution to the technical problem of automatically verifying functionality of the neurofeedback headset used to administer neurofeedback to a patient using multiple sessions. The functionality of the neurofeedback headset may be tested before and/or after each session, to help verify the ability of the neurofeedback headset to measure brain signals correctly.

The systems and/or methods described herein tie mathematical operations (e.g., analyzing sensed signals generated by the cradle) to the ability of a processor to execute code instructions, for example, by analyzing the sensed signals to verify functionality of the neurofeedback headset.

The systems and/or methods described herein improve performance of a system that applies neurofeedback treatments (e.g., neurofeedback headset measuring brain signals, and/or computing unit responsive to the measured brain signals that adjusts video and/or audio presented to the patient), by providing a cradle that is used to test the functionality of the neurofeedback headset measuring brain signals (e.g., EEG) as part of an automatic administration of neurofeedback treatment. Errors in the neurofeedback headset may be quickly and/or easily detected (and corrected) before and/or after each treatment.

Accordingly, the systems and/or methods described herein are necessarily rooted in computer technology to overcome an actual technical problem arising in the technical field of neurofeedback therapy.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages.

The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

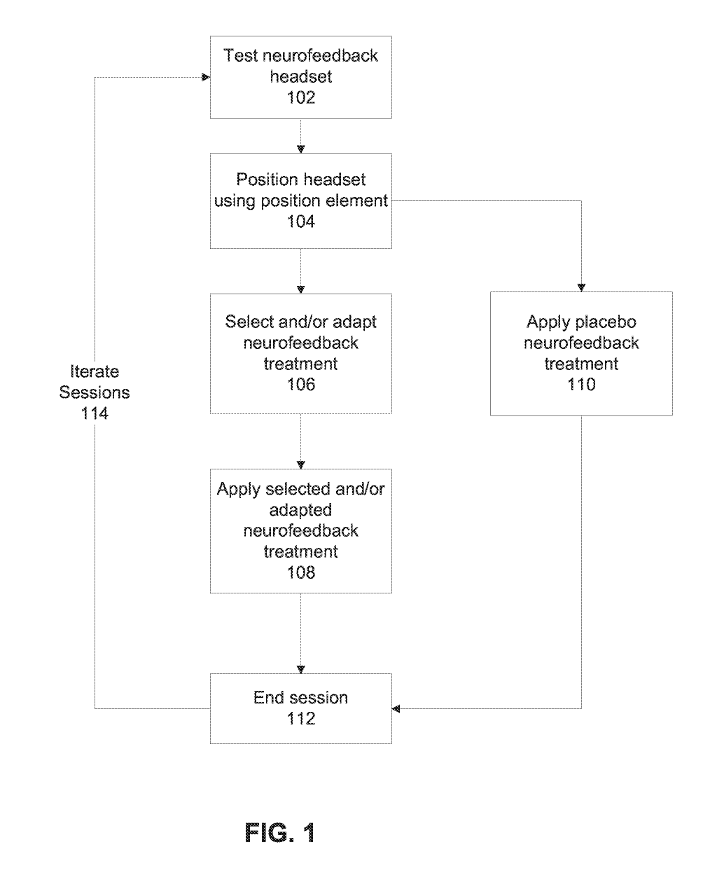

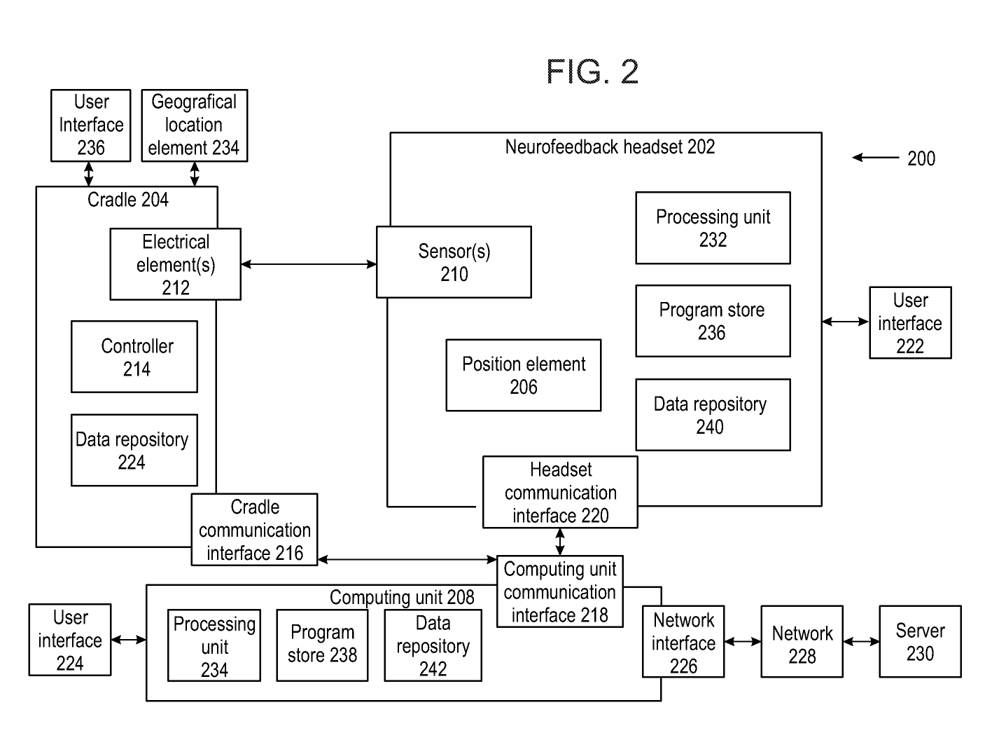

Reference is now made to FIG. 1, which is a flowchart of a method for automatically administering neurofeedback treatment (optionally in multiple sessions) to a patient, in accordance with some embodiments of the present invention. The method includes one or more of: verifying functionality of a neurofeedback headset, positioning the neurofeedback headset on the head of the patient based on a predefined location, dynamically selecting and/or adjusting the neurofeedback treatment for administration to the patient, and administration of a placebo neurofeedback treatment. Reference is also made to FIG. 2, which is a diagram of components of a system 200 that automatically administers neurofeedback treatment to a patient, in accordance with some embodiments of the present invention. The acts of the method of FIG. 1 may be implemented by components of system 200. For example, the functionality of a neurofeedback headset 202 may be verified using a cradle 204. Neurofeedback headset 202 may be positioned using position element 206. The neurofeedback treatment may be selected and/or adapted by computing unit 208. The placebo treatment may be administered by computing unit 208.

Neurofeedback headset 202 includes sensors 210 that sense brain signals, optionally electrodes to sense EEG signals. Optionally, the electrodes are dry electrodes. Neurofeedback headset 202 may include a brain signal processing system that includes one or more of: an audio system (e.g., audio headset, such as speakers), signal (e.g., radio wave) amplifier, and analogue to digital converter.

Different neurofeedback headsets 202 may be designed to treat different conditions, for example, varying by number, position, and/or type of electrodes, and/or code instructions stored in program store 236. For example, 5 electrodes may be used to improve memory. Neurofeedback headset 202 may be available in different sizes for different heads and/or be adjustable to fit different head sizes. Neurofeedback headset 202 may be made from a light material (e.g., plastic, foam, aluminum) and/or include multiple orifices for aeration and weight reductions. Neurofeedback headset 202 may or may not include a face strap.

Cradle 204 includes a controller 214 (e.g., processor executing code stored in a program store, and/or circuitry such as a FPGA) that controls electrical elements 212 to generate test signal(s) for reception by sensors 204 of neurofeedback headset 202 during a testing process, as described herein.

Cradle 204 may include a cradle communication interface 216 for communicating with computing unit 208. Computing unit 208 includes a computing unit communication interface 218 for communicating with cradle 204 and/or neurofeedback headset 202 via respective cradle communication interface 216 and headset communication interface 220. Exemplary interfaces 216, 218, and/or 220 include, for example, a wire and/or wireless communication interface, for example, a short range wireless interface, a network interface, a cellular interface, a cable interface (e.g. universal serial bus (USB)), and/or a virtual interface.

Cradle 204, computing unit 208, and/or neurofeedback headset 202 may be powered and/or charged by batteries (optionally rechargeable), a wall outlet (via a cable and plug, which may include connectors to plug into different outlets across the world), a USB cable, microUSB connectors, or other methods.

The battery may provide power, for example, for up to 10 hours. Neurofeedback headset 202 may be charged via cradle 204, when positioned on cradle 204.

Neurofeedback headset 202 and/or computing unit 208 may include or be in communication with respective user interfaces 222 and 224 that allows the patient (or other user) to enter data and/or provides the patient (or other user) with outputted data, for example one or more of: a touch-surface such as: a keyboard, a touch-pad, a touch-screen, button(s), dial(s), and a touch-screen, and/or a microphone (with optional voice recognition software), and/or speaker.

Computing unit 208 may include or be in communication with a network interface 226 (e.g., wired and/or wireless connectivity) that connects computing unit 208 to a network 228, for example, a wireless network, a cellular network, the internet, and a local area network. Computing unit 208 may communicate with one or more remote servers 230 over network 228, for example, to download updated and/or new neurofeedback treatments which may be used in the process of selecting the effective neurofeedback treatment, as described herein. Server 230 may perform centralized upgrades and/or maintenance of computing units 208, neurofeedback headsets 202, and/or cradles 204. Computing unit 208 may be programmed to execute applications according to instructions verified as originating from server 230 (e.g., without enabling other applications).

Computing unit 208 may be implemented as a mobile device, for example, a smartphone, a tablet computer, a laptop, and a wearable device (e.g., computing lasses, watch computer). Computing unit 208 may be implemented as a remote server, a desktop computer, a dedicate device, a web server, or other computing units (e.g., over a suitable communication connection between neurofeedback headset 202 via interface 220 or another interface).

Computing unit 208 may include code instructions (stored in program store 238 executing by processing unit 234) to manage patient records (e.g., create new patient records, store patient data, and manage patient data), and/or communicate with remote server 230 (e.g., to backup patient data, which may allow research to be performed based on data from different patients).

Processing unit 232 of neurofeedback headset 202 and/or processing unit 234 of computing unit 208 (and/or processing unit of cradle 204 when controller 214 is implemented to include the processing unit) may be implemented as, for example, a central processing unit (CPU), a graphics processing unit (GPU), field programmable gate arrays (FPGA), digital signal processor (DSP), and application specific integrated circuits (ASIC). Processing unit 232 and/or 234 may include one or more processors (homogenous or heterogeneous), which may be arranged for parallel processing, as clusters and/or as one or more multi core processing units.

Program store 236 of neurofeedback headset 202 and/or program store 238 of computing unit 208 (and/or program store of cradle 204 when controller 214 is implemented to execute code stored in the program store) store code implementable by respective processing units 232 or 238, for example, a random access memory (RAM), read-only memory (ROM), and/or a storage device, for example, non-volatile memory, magnetic media, semiconductor memory devices, hard drive, removable storage, and optical media (e.g., DVD, CD-ROM).

Data repository 240 of neurofeedback headset 202 and/or data repository 242 of computing unit 208 and/or data repository 244 of cradle 204 may be implemented, for example, as a hard drive, removable storage, built-in storage, a remote server, and/or other storage devices. Respective data repositories may store, for example, activation patterns for transmitting via electrical elements 212 for testing neurofeedback headset 202 (e.g., stored in data repository 244 of cradle 204), and a dataset of neurofeedback treatments for selection (e.g., stored in data repository 242 of computing unit 208).

The systems and/or methods (e.g., implemented by a processor executing code instructions stored in a memory) described herein may be used by patients for self-treatment, without necessarily requiring a doctor or therapist for supervision of the session. Patients may self-administer neurofeedback therapy at home, as compared to a clinical setting. Alternatively or additionally, the systems and/or methods described herein may be set-up and/or initialized in a professional setting (e.g., senior home, general medical clinic, community center, private clinic) by a technician (or amateur) with minimal training, for example, to help the patient with the initial system setting that will allow the patient to use the system at home, and to provide initial training to patients on how to use the system at home. The patient may then continue treatment at home, for example, to self administer the effective neurofeedback treatment.

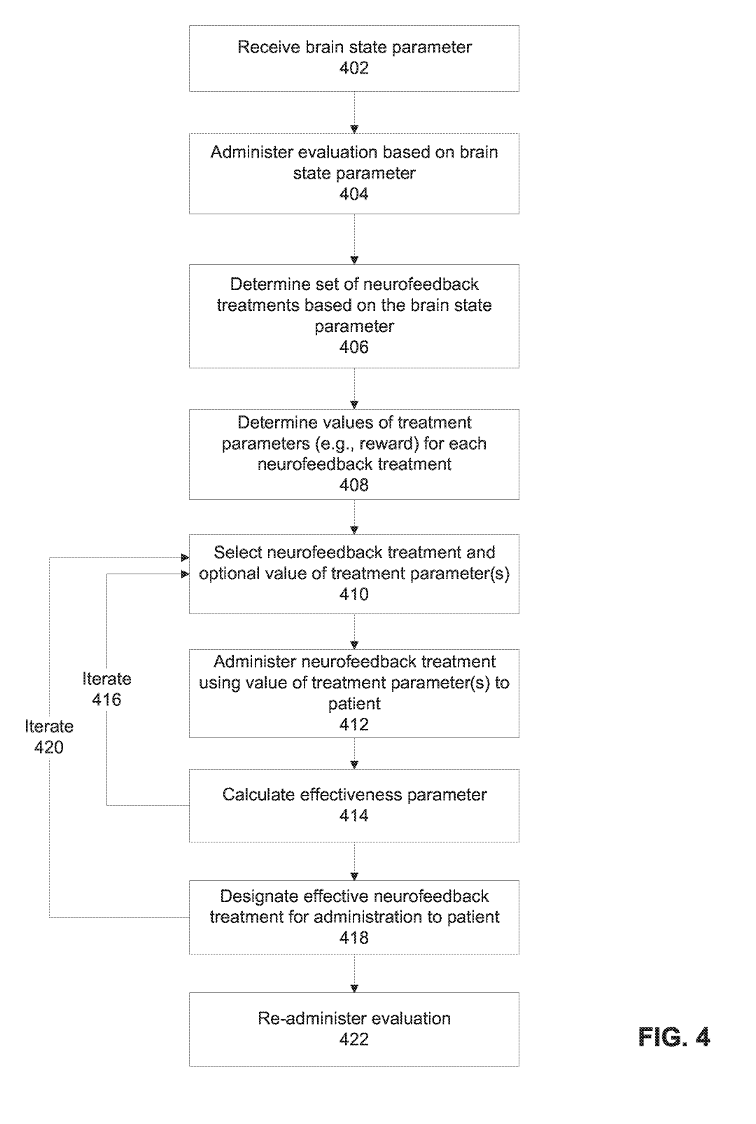

Optionally, system 200 is designed for use in a professional setting (e.g., clinic, senior home, community center) for administration by a trained technician. For example, code stored on program store 238 of computing unit and/or on program store 236 of neurofeedback headset may include code to determine the effective neurofeedback therapy (e.g., as described with reference to FIG. 4). The professional setting may help patients get accustomed to the treatment, while a professional oversees the initialization of the system, for example, using a suitable GUI presented on user interface 224. The determined effective neurofeedback treatment may be administered at home or in the professional setting. Another implementation of system 200 may be designed for home use by the patient, without necessarily requiring professional intervention, for example, by including code instructions to perform one or more of the acts described with reference to FIG. 1. The professional and home versions may differ in design (e.g., positioning of electrodes, number of electrodes) or be similar in design. The professional and home versions may differ in the code instructions stored in respective program stores of the computing unit and/or headset, or may store similar code. Alternatively, the home and professional implementations are the same or similar, allowing for home and/or professional use.

In an example, when the patient obtains help from a technical to set-up system 200, the selected parameters (e.g., determined effective neurofeedback treatment and/or treatment plan) and/or other patient data may be stored and transferred from the computing unit of the technician to the computing unit and/or neurofeedback headset of the patient, for example, over a wireless connection, using a removable memory device (e.g., USB stick), and/or downloaded over network 228. The removable memory device may be used for identifying of the user, for example, the neurofeedback treatment is automatically initialized and set-up when the user inserts the USB stick into neurofeedback headset 202 and/or computing unit 208.

The removable memory stick may be used, for example, by patients who might not have an easy/available internet connection, have difficulty in remembering their passwords, and/or who might not want the hassle to perform login, yet still enjoy a completely automatic therapy option.

Alternatively, user data may be validated before treatment (e.g., when the user logs in using a GUI presented on user interface 224 of computing unit 208) and/or treatment related data may be downloaded from server 230 (e.g., operated by the clinic that performed the initialization process) over network 228.

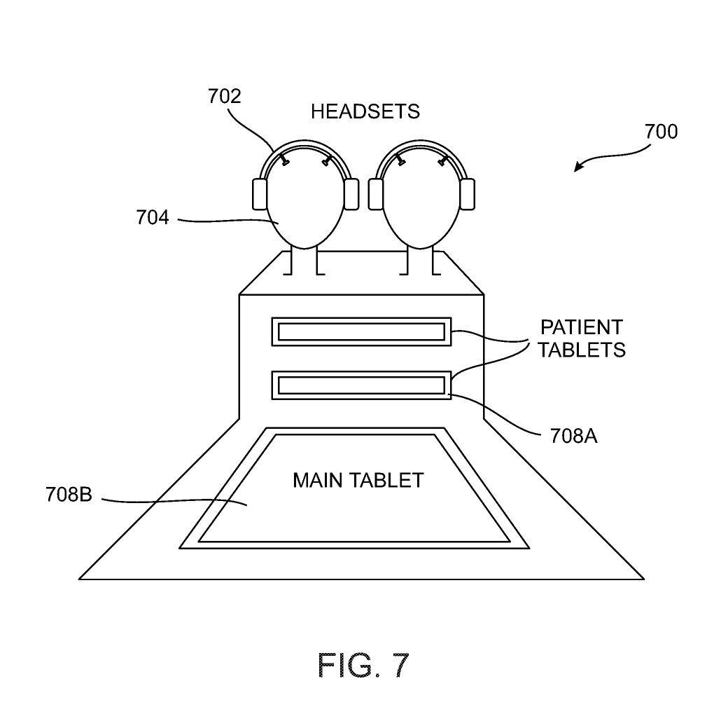

Reference is now made to FIG. 7, which is a schematic of an example of a professional implementation 700 of system 200 of FIG. 2, in accordance with some embodiments of the present invention. Professional system implementation 700 may include one or several neurofeedback headsets 702, for example, to simultaneously treat different patients, and/or each headset designed for different treatments (e.g., varying by electrode placement and/or electrode design). Headsets 702 may rest on respective cradles 704 (as described herein). Each patient may receive a patient computing unit 708A, which may present video and/or audio that is adapted based on measured brain signals (and/or delivers a placebo), as described herein. An operator computing unit 708B may be used by a technician or other professional administering the treatment, for example, to supervise the administration of the treatment.

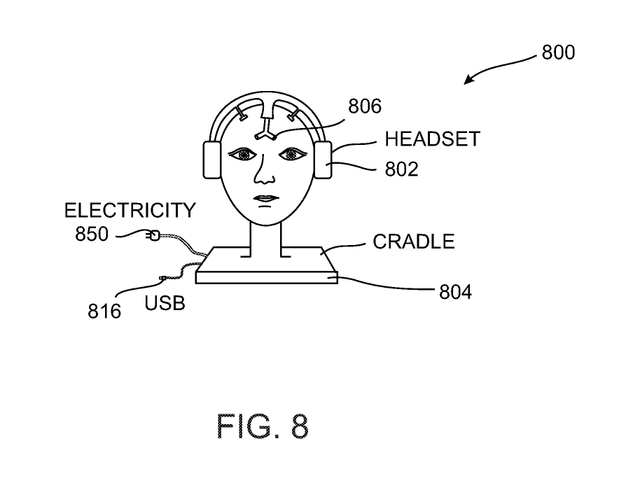

Reference is now made to FIG. 8, which is a schematic of an exemplary of a home implementation 800 of system 200 of FIG. 2, in accordance with some embodiments of the present invention. Neurofeedback headset 802 is positioned on cradle 804, optionally guided into the correct position by position element 806 (as described herein). Cradle 804 may include a USB connector 816 to communicate with computing unit 208, and/or an electrical plug 850 to provide electricity to cradle 804 and/or for charging batteries of neurofeedback headset 802.

Program store 238 of computing unit 208 and/or program store 236 of neurofeedback headset 202 may store code instructions defining neurofeedback parameters for delivery of neurofeedback treatments, for example, the defined effective neurofeedback therapy (e.g., selected as described herein), preferred video and/or images to present as part of the neurofeedback treatment and/or preferred audio to sound as part of the neurofeedback treatment, preferred video and/or audio content for the treatment, reminders generated based on a treatment schedule (e.g., transmit a message to a Smartphone of the user, beep or flash a light indicating time for a treatment session), and configuration parameters (e.g., obtain patient data from external storage unit, such as the USB stick).

System 200 may be operated in automatic mode, based on the stored parameters, and/or in manual mode, in which the user logs in and manually defines the parameters (e.g., using the GUI presented on display user interface 224). For example, to operate in automatic mode, the user may insert the external storage device into a slot of computing unit 208 to trigger operation without necessarily requiring human intervention (e.g., based on one or more of the acts of the method of FIG. 1).

Optionally, neurofeedback code (e.g., one or more functions as described herein) stored in program store 238 of computing unit 208 is implemented as an application that may be installed on Tablets, Smartphones, laptops, mobile devices, or other computing units 208, for example, downloaded from server 230 over network 228, and/or installed from storage (e.g., CD, external storage unit). The neurofeedback code may store patient statistics (e.g., patient demographics, success of treatments) locally (e.g., in data repository 242) and/or transmit the patient statistics to server 230. Neurofeedback code may be designed to execute within standard off-the-shelf operating systems. Multi language support may be provided using the GUI.

Neurofeedback code may access short movies (e.g., 20-30 minutes long, which may match the length of the neurofeedback session) for adaptation as part of the neurofeedback treatment. The movies may reside on a remote server, and/or locally in data repository 242. The movies may be selected based on user theme preferences (which may be stored as user parameters), for example, nature, TV series, and comedy.

Neurofeedback code access a simple game for use in the neurofeedback session that responds based on brain signals generated by the brain of the patient. For example, when the user is focused, a big truck begins to move on a road. When the user is not focusing, the truck stops. The games may be stored locally and/or on the remote server.

Referring back to FIG. 1, at 102, the function of neurofeedback headset 202 is automatically verified by computing unit 208 based on signals generated by cradle 204. The functionality of neurofeedback headset 202 may be tested before each neurofeedback session, or selected sessions, for example, every 5 or 10 sessions, or every newly selected treatment. Errors in neurofeedback headset 202 may be automatically detected before the session, to help ensure that the neurofeedback session is correctly delivered using a functional neurofeedback headset 202.

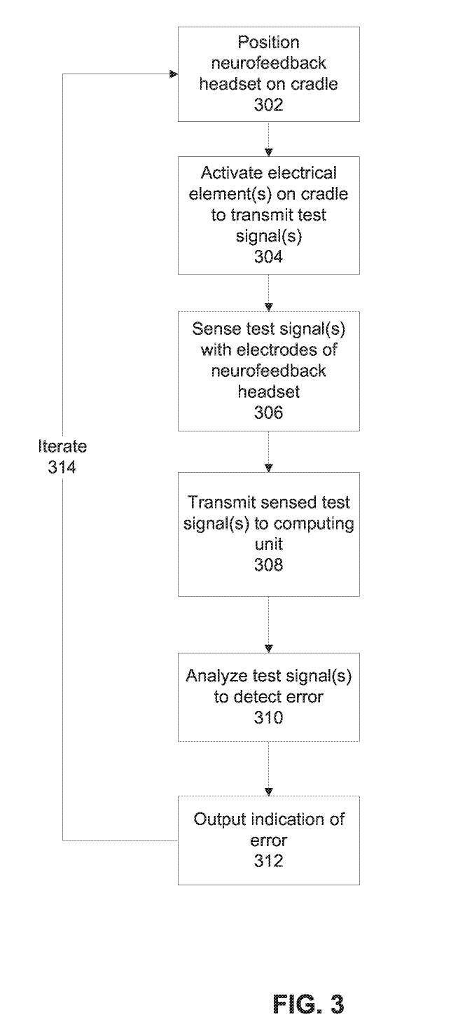

Reference is now made to FIG. 3, which is a flowchart of a method for automatically verifying function of a neurofeedback headset, in accordance with some embodiments of the present invention. Reference is also made to system 200 of FIG. 2, which may implement the automatic testing of neurofeedback headset 202. The automated text process may verify, for example, calibration of sensors 210 and/or of the neurofeedback headset 202, basis functions of sensors 210, correct processing of received signals by processing unit 232 of the neurofeedback headset 202, and/or other functions. The method allows users to administer neurofeedback treatments themselves, without requiring a trained technician or therapist to verify that the equipment is working properly.

At 302, neurofeedback headset 202 is positioned (or already located) on cradle 204. Cradle 204 includes a surface shaped to accommodate an inner surface of neurofeedback headset 202 (i.e., that contacts a head of a patient when the patient wears neurofeedback headset 202). The surface is shaped according to the size and shape of the head of the patient. The surface has a shape similar at least to the top of the head (i.e., above the circumference of the eyes and ears, including the portion where hair grows on top of the head). The surface may be custom made, or selected from multiple available cradle designs according to the size and/or shape of the head of the patient.

Electrical elements 212 are arranged along the surface of cradle 204 (e.g., below the surface, embedded within the surface, or coupled above the surface) to correspond to the location of sensors 210 of neurofeedback headset 202. When neurofeedback headset 202 is positioned on the surface of the cradle 204, sensors 210 (e.g., EEG electrodes) of neurofeedback headset 202 are in electrical communication with respective electrical elements 212 of the surface of cradle 204.

Optionally, the electrical elements 212 are transmitters (and/or transceivers) designed to create electrical communication by physically contacting sensors 210 (e.g., EEG electrodes) of neurofeedback headset 202 when neurofeedback headset 202 is correctly positioned on the surface of the cradle 204. Electrical signals are conducted via the physical connection.

Alternatively or additionally, electrical elements 212 are electromagnetic generators designed to create wireless electrical communication with sensors 210 (e.g., electrodes) of neurofeedback headset 202 without necessarily physically contacting sensors 210 when neurofeedback headset 202 is positioned on the surface of cradle 204. Electrical signals are conducted wirelessly. Testing based on the wireless signal communication may not necessarily require accurate positioning of neurofeedback headset 202 on cradle 204.

Neurofeedback headset 202 may include a position element 206 shaped to help correctly position neurofeedback headset 202 on cradle 204, for example, when cradle 204 is shaped similar to the head of the patient, including similar facial features of the patient, such as eyes and nose. Position element 206 is described herein in greater detail.

The testing process may be automatically triggered when neurofeedback headset 202 is positioned correctly on cradle 204.

At 304, electrical elements 212 of cradle 204 are activated to generate signals for sensing by sensors 210 of neurofeedback headset 202. Signal generation may be controlled by controller 214 according to a predefined pattern which may be stored in data repository 244. For example, the predefined pattern may include activating electrical elements 212 to simulate EEG signals which may be similar to EEG signals generated by the brain of the patient. In another example, controller 214 activates electrical elements 212 to generate impedance signals to verify electrical conductivity and/or basic functionality of sensors 210 and/or within neurofeedback headset 202.

Controller 214 may activate electrical elements 212 one at a time, several at a time, or all at a time. The pattern of activation may be determined according to the function being tested. For example, all electrical elements 212 may be activated when simulating EEG signals. In another example, each electrical element 212 may be activated one at a time to avoid interference with other electrical elements 212, for example, when performing a check of calibration.

Controller 214 may activate electrical elements 212 according to instructions received from computing unit 208. The instructions may be received and executed in real-time, or received and stored by cradle 204. Instructions may be received via cradle communication interface 216 communicating with computing unit communication interface 218.

At 306, signals generated by electrical elements 212 are sensed by sensors 210 of neurofeedback headset 202. The sensed signals may undergo processing by neurofeedback headset 202 (e.g., by processing unit 232), for example, analogue to digital conversion, filtering, amplification, or other signal processing.

At 308, the sensed signals (e.g., post processing) may be transmitted by neurofeedback headset 202 to computing unit 208, optionally via headset communication interface 220 to computing unit communication interfaced 218.

At 310, the sensed signals (e.g., post processing) are analyzed by computing unit 208 (or at neurofeedback headset 202). The signals are analyzed to verify correct functionality of neurofeedback headset 202. The signals may be analyzed by correlating the received signals to expected signals according to a correlation requirement representing an allowable tolerance within which proper functionality is maintained. For example, computing unit 208 may instruct cradle 204 to generate a simulated EEG signal at one or more electrical elements 212, in real-time or based on stored instructions. Computing unit 208 may correlate the received sensed signals with the instructions simulated EEG signals to determine whether the received signals are correlated with the generated simulated EEG signals according to the correlation requirement.

At 312, when an error is detected, for example, the received sensed signals do not correlate with the simulated signal, an output indicative of the error is generated.

The output may be presented to the user using user interface 224 and/or user interface 222, for example, a blinking red light, a message presented within a graphical user interface (GUI) on a display. Details of the analysis may be stored in data repository 242 to assist in fixing the error, for example, which sensor 210 experienced the error, and the nature of the error.

At 314, one or more blocks 304-312 are iterated, for example, to conduct multiple tests. Each test may test, for example, different sensor(s) 210, administer different signal patterns (e.g., different EEG signals), signal processing integrity of neurofeedback headset 202 in processing the sensed signals, and different functions (e.g., connectivity, signal reception sensitivity, noise levels).

Referring now back to FIG. 1, at 104, the patient positions neurofeedback headset 202 on their head at a predefined position. When headset 202 is positioned at the predefined position, sensors 210 of neurofeedback headset 202 are positioned relative to locations on the head of the patient that are suitable for sensing brain signals (e.g., EEG). Position element 206 of headset 202 is designed to help the patient repetitively position headset 202 at the predefined position to allow repetitive measurements of the brain signals at the same or similar locations on the head. The patient may repetitively position headset 202 at the predefined position at each treatment session, and/or during the treatment session itself, such as to reposition headset 202 when headset 202 slips out of the predefined position.

The neurofeedback code may assist the user in validating that neurofeedback headset 202 is properly communicating with computing unit 208, for example, by receiving initialization signals transmitted by neurofeedback headset 202 using headset communication interface 220. A GUI presented on display (user interface 224) may present an image representing the headset positioned on the head of the patient. Functionality of sensors 210 may be tested when neurofeedback headset 202 is positioned on the head of the patient, for example, to verify proper contact with the head of the patient and/or the correct reading of brain signals (e.g., based on a signal to noise requirement). Properly functioning electrodes may be colored in green on the image of the GUI. Electrodes not correctly measuring EEG signals may be colored in red or grey. Treatment may automatically start when all electrodes are in green. A troubleshooting guide or wizard may be trigged when one or more electrodes are in red or grey to help the user solve the problem.

In another example, a camera in communication with computing unit 208 (e.g., user interface 224) may capture one or more images of the patient wearing headset 202. Image processing code (e.g., stored in program store 238) may analyze the image to determine whether headset 202 is positioned at the predefined position on the head of the patient. The image analysis may be used to help guide the patient to the proper position (e.g., generating instructions on how to proceed from the current position of headset 202 to the predefined position, for example, rotate the headset clockwise, tip headset forward). The image analysis image may first capture image(s) of the patient without headset 202. The images may be analyzed (e.g., using image processing code) to determine the anatomy of the head of the patient, for example, sizes and/or shape of the frontal bone, width of eyes, shape of the nose, and relative positioning of eyes relative to nose. The image analysis maybe used to verify the correct position of the headset 202 at the predefined position. Multiple images at different viewing angles may be captured and analyzed, for example, a face on view of the head of the patient, a side view of each side, and a back view.

At first usage, neurofeedback code may register the serial number of neurofeedback headset 202 (e.g., which may be automatically transmitted from neurofeedback headset 202 to computing unit 208 using respective interfaces 220 and 218, optionally based on a query transmitted by the neurofeedback code of computing unit 208). Neurofeedback code may transmit the serial number to server 230 over network 228 in association with the user credentials as part of the registration process. Neurofeedback code may present information associated with the registration process within the GUI presented on display (user interface 224), for example, explaining that the registration is required to obtain the following features: warranty, user data backup (e.g., for changing computing units 208), tracking user progress (e.g., generating reminders), and automatic updates of the neurofeedback code, movies used for the treatment, and/or new neurofeedback treatments.

Optionally, position element 206 guides the positioning of neurofeedback headset 202 on the head of the patient.

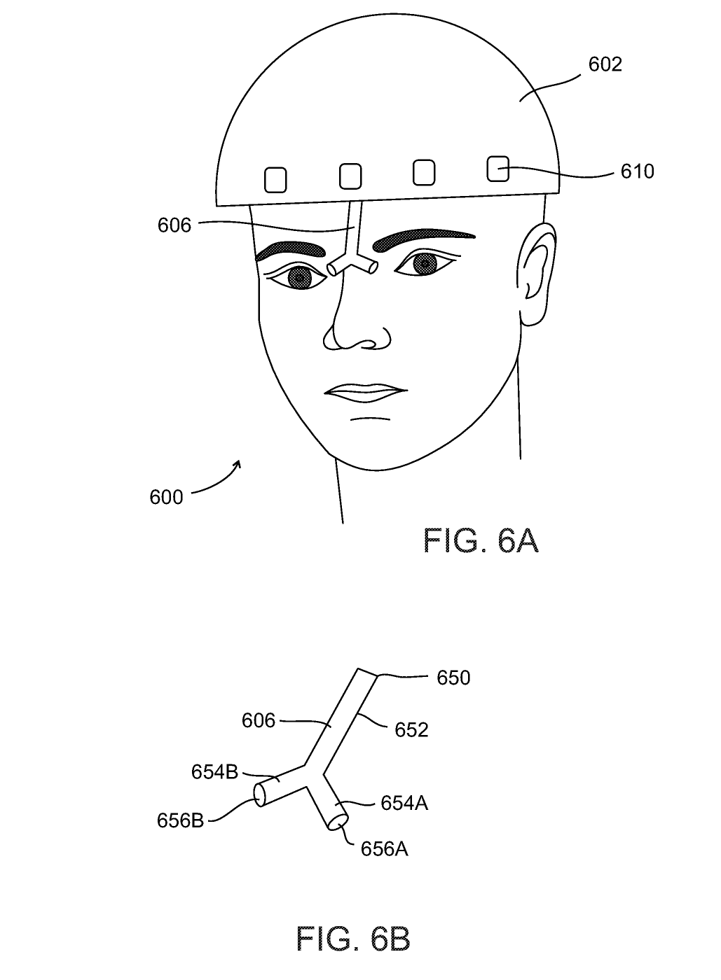

Reference is now made to FIG. 6A, which is a schematic depicting a position element 606 for positioning a neurofeedback headset 602 at a predefined location on the head of a patient, in accordance with some embodiments of the present invention. Reference is also made to FIG. 6B, which is a blow-up of position element 606 (shown alone), in accordance with some embodiments of the present invention. Neurofeedback headset 602 includes sensors 610 (e.g., EEG electrodes) that output brain signals (e.g., EEG) when contacting the head of the patient when neurofeedback headset 602 is positioned at the predefined position using position element 606. Position element 606 allows the patient to repeatedly position neurofeedback headset 602 at similar positions on the head (e.g., within an allowable tolerance requirement).

The predefined position allows for multiple neurofeedback sessions to be administered correctly based on proper EEG measurements without necessarily requiring re-calibration.

Position element 606 includes a first end portion 650 for coupling to the anterior and inferior portion of the neurofeedback headset 602, such as a lower edge of the front of neurofeedback headset 602 centered between the eyes. The coupling may be performed for example, by clicking into a slot in neurofeedback headset 602, using glue, or velcro, or by manufacturing as an integral part of neurofeedback headset 602 (e.g., using injection molding).

An elongated portion 652 extends inferiorly from first end portion 650. The length of elongated portion 652 is selected such that when neurofeedback headset 602 is located at the predefined positioned on the head of the patient, elongated portion 652 extends approximately parallel to the surface of the frontal bone of the patient, optionally until the glabella of the patient, for example, a length of about 3 centimeters, of about 5 centimeters, or about 7 centimeters, or other lengths. Optionally, the length of elongated portion 652 is selected according to the size and/or shape of the frontal bone of the patient. Alternatively or additionally, the length of elongated portion is adjustable 652 to fit patients having different frontal bone surface sizes, for example, using a screw and bolt mechanism, using a stopper mechanism, using velcro, or handle with extension mechanism.

Each member of a pair of arms 654A-B extends laterally in opposite directions and inferiorly from the end portion of elongated portion 652 (positioned at the glabella when worn by the patient). Each respective arm 654A-B is oriented for positioning along the respective side of the nasal bone and/or inferiorly to the respective eyebrow and superiorly to the respective eye of the patient.

Each arm 654A-B may include a contact element 656A-B at its respective end portion designed to contact the skin, optionally without exerting sufficient pressure to damage the skin (e.g., pressure sore). Each contact element 656A-B is sized to be small enough to avoid contact with the eye of the patient when at the predefined position.

The end portion (e.g., contact element 656A-B) of each arm of the pair of arms is positioned medially to the respective supraorbital notch of the patient.

Optionally, elongated portion 652 is positioned and/or biased to contact the skin surface of the frontal bone. The entire (or most) of the length of elongated portion 652 may contact the skin. Alternatively, elongated portion 652 is designed to avoid contact with the skin, the skin being contacted by contact elements 656A-B and/or by the region of elongated portion 652 that splits to form arms 654A-B.

Position element 606 may be custom manufactured for each patient, selected from a set of available position elements having different sizes, and/or adjusted according to the patient.

Position element 606 may be made from a rigid material, for example, plastic and/or metal. Position element 606 may be made from a material that allows some yielding, for example, to allow some bending and shape deformation to accommodate different facial anatomies of patients.

One or more of elements 652, 654A, 654B, 656A, 656B, may be designed to be of variable adjustable length. The length may be adjusted to fit the anatomy of the patient. Exemplary mechanisms for length adjustment include, using friction, pre-selectable length choices, telescopic tube, and cheese tube and handle.

A tutorial (e.g., stored as code instructions in data repository 242 of computing unit 208) which may be presented within a GUI on a display (e.g., user interface 224) may guide to the patient to proper placement of position element 606 on their head.

The tutorial presented within the GUI may instruct the patient on adjusting components of position element 606 to fit the anatomy of the patient's face and/or head, for example, adjust elongated portion 652 to the appropriate size for the face of the patient, and/or increase or decrease the distance between contact elements 656A-B according to the anatomy of the patient. Alternatively or additionally, the initial set-up of position element 606 (e.g., adjustment of length according to the face of the patient) is performed by a trained technician at a clinic.

The tutorial may be executed in block 104 of FIG. 1. Referring now back to FIG. 1, at 106, a neurofeedback treatment is selected for administration to the patient. Alternatively or additionally, the currently administered neurofeedback treatment (or the selected neurofeedback treatment) is adjusted, for example, by adjusting the reward threshold (i.e., the requirement to adapt the image, video, and/or audio the user is watching or listening to based on calculations performed on sensed brain signals). It is noted that when the patient is listening (e.g., to a soundtrack), the neurotherapy treatment may be applied when the patients eyes are open or closed.

Reference is now made to FIG. 4, which is a flowchart of a method for selecting and/or adapting a neurofeedback treatment for patient, in accordance with some embodiments of the present invention. The method may be implemented by system 200 described with reference to FIG. 2.

The method adapts neurofeedback treatments and/or selects optimal neurofeedback treatment(s) according to the brain of the patient instead of, for example, applying the same generic treatment to different patients and/or the treatment depending on the therapist selected by the patient.

Selected treatments may be adapted based on the current state of the brain of the patient, and/or new treatments may be selected based on the current state of the brain of the patient. In this manner, the neurofeedback treatment is customized to the patient and adapted according to the changing brain of the patient. The method select successful treatments that are more efficient for delivery during a shorter treatment time. The method may monitor the effectiveness of the selected neurofeedback treatment on the brain of the patient, and/or adapt the selected treatment when the monitoring determines that the effeteness is reduced.

The method may improve memory functions in patients with mild cognitive impairment (e.g., amnestic MCI) which may be a prodromal stage of Alzheimer's disease, and/or delay or prevent deterioration into Alzheimer's, by selecting and adapting the best neurofeedback treatment for each patient. The method may delay or stop the progression from MCI to Alzheimer or other dementia by selecting the best neurofeedback treatment for each patient.

The method may improve executive functions in patients diagnosed with ADD or ADHD, and improve the attention capabilities of the patients. The method may improve peak performance of students. The method may improve peak performance of athletes. The method may be used to treat autism.

The efficiency of neurofeedback treatments may be increased, allowing patients to obtain better results faster. A greater number of patients may benefit from neurofeedback treatments, by customizing the treatment per patient. The method may automatically select and/or adapt neurofeedback treatments without necessarily requiring human intervention before and/or during the neurofeedback treatment session. The patient may easily and automatically self-administer the testing process and the designated neurofeedback treatment.

At 402, one or more patient brain state parameters indicative of a current brain state of a patient for treatment using neurofeedback is received by computing unit 208, for example, manually entered by a user and/or operator using user interface 224 (e.g., using a GUI presented on a display which may present an intake questionnaire), automatically calculated (e.g., based on an electronic medical record of the patient), and/or retrieved from storage (e.g., from a database storing patient data.

The brain state parameter may represent a current state of the brain of the patient.

The brain state parameter may include a sign and/or symptom, independent of underlying cause. For example, the brain state parameter may include memory improvement, improvement in executive function, and/or attention improvement. The brain state parameter may indicate the current state of the brain of the patient independent of underlying cause, for example, memory improvement due to mild cognitive impairment, secondary to a stroke, secondary to drug or alcohol abuse, or an improvement desired by a healthy patient (e.g., student studying a profession requiring memorization of a large amount of material).

Alternatively or additionally, the brain state parameter is indicative of the pathology or cause of the signs and/or symptoms, for example, mild cognitive impairment (e.g., related to Alzheimer' s or other dementia such as vascular dementia), attention disorders (e.g., ADD, ADHD), epilepsy, post-stroke patients, psychiatric patients, brain injury, and adverse effects due to long term drugs and/or alcohol abuse.

At 404, an evaluation to obtain a baseline score for the patient at the current brain state of a patient may be administered. The evaluation may be administered manually (with results entered into computing unit, for example, using the GUI presented on display of user interface 224) and/or automatically (e.g., presenting the evaluation on the GUI and asking the user to perform tasks or enter answers).

The evaluation may include an analysis of an implementation of system 200 selected by the patient, for example, the patient may purchase neurofeedback headset 202, computing unit 208, and/or the code instructions to install on headset 202 and/or computing unit 208. For example, when the patient selects system 200 designed for memory enhancement, the evaluation determines that the patient wants to be treated for memory enhancement. For example, when the patient selects system 200 designed to improve peak performance, the evaluation determines that the patient wants to be treated for peak performance. The evaluation may be performed automatically, for example, by code instructions checking which software implementation is installed, and/or manually by the user, for example, by scanning a barcode indicating the implementation of system 200, and/or manually entering using a GUI the serial number of system 200 and/or other indication of the implementation.

The evaluation may include a validated scoring tool used for patients with medical condition, for example, the mini mental status exam (MMSE) for patients with cognitive impairment. The evaluation may include a validated memory test scoring tool, for example, the Recognition Memory Test, the Coughlan and Hollows Information Processing Test, the Californian Verbal Learning Test, and the set of five Camden memory tests. The evaluation may include a non-validated scoring tool, for example, a simple memory exam, for example, random numbers (with increase in the number of digits) are presented for the user to memorize. The evaluation may include a computerized test performed without necessarily requiring explicit user entered data, for example, a quantitative EEG performed using the neurofeedback headset.

The score based on the evaluation may be included in the brain state parameter, for example, the score of the MMSE may be included in the brain state parameter, or the number of digits the user is able to memorize.

The evaluation may include a baseline set of measured brain signals, for example, a baseline of EEG measurements, performed with open and/or closed eyes.

At 406, the patient brain state parameter(s) is correlated with a set of neurofeedback treatments from multiple neurofeedback treatments stored in a dataset, for example, in data repository 242, or on a remote server 230 (accessed by computing unit 208 over network 228).

The neurofeedback treatments may be stored, for example, in a database, as individual records, or based on textual tags.

The brain state parameter may be matched to multiple neurofeedback treatments, for example, using a look-up table, and/or a mapping function. For example, when the brain state parameter includes memory improvement, the look-up table or mapping function may be used to identify neurofeedback treatments suitable for memory improvement, for example, the neurofeedback treatments are labeled using a memory improvement tag, or the look-up table maps the memory improvement query to database entries of neurofeedback treatments.

The brain state parameter may be correlated to multiple neurofeedback treatments, for example, using a regression function, statistical classifier, or other machine learning methods, for example, to select neurofeedback treatments for a patient with mild cognitive impairment related to onset of Alzheimer's and an MMSE score of 22.

For example, when the patient brain state parameter includes memory improvement, the correlated and/or matched set of neurofeedback treatments include one or more of: absolute power value of the Alpha frequency measured at a selected electrode, relative power of the Alpha frequency compared to the power of the rest of all other frequencies measured for the selected electrode, average power of the measured Alpha frequency over time, and coherence between the phases of the Alpha frequency of several electrodes. One or more of the listed treatments may be applied to determine the most efficient treatment, as described herein. It is noted that the list of neurofeedback treatments is exemplary, and not meant to be exhaustive, as other neurofeedback treatments may be applied using the systems and/or methods described herein.

At 408, each member of the set of neurofeedback treatments is associated with treatment parameters. The values of the treatment parameters that may be assigned to each neurofeedback treatment may be stored, for example, in a database, as a set of records, or calculated by a function, for example, stored in data repository 242 and/or on remote serer 230 accessed over network 228. Each neurofeedback treatment is associated with multiple different values for a requirement target of the treatment parameter. A calculation based on outputs of sensor(s) 210 measuring brain signals of the patient is compared to the value of the requirement target, for example, as a threshold, as a range, or as a function.

As used herein, the term set of neurofeedback treatments may include the combination of each treatment with different values of treatment parameters (e.g., the same treatment administered using different reward value thresholds).

Optionally, the treatment parameter includes a reward requirement (e.g., range, threshold) defining adaptation of images, video, and/or audio according to measurements of brain signals (e.g., based on EEG signals) of the patient. For example, the treatment parameter defines a threshold at which a video the patient is watching is darkened or presented normally (or varying the quality level when a function is used).

Each neurofeedback treatment includes different values for the reward requirement (e.g., threshold). For example, the absolute power value of the Alpha frequency measured at a selected electrode is associated with a 50% threshold, a 60% threshold, and a 75% threshold, creating three possibilities.

Dynamic selection of the rewards may improve matching of the neurofeedback therapy to the brain of the patient, for example, since some brains might learn better when given numerous easy to obtain rewards, while other brains might learn better when the treatment provides them with fewer and tougher to obtain rewards.

The combination of neurofeedback treatments and treatment parameters (e.g., reward requirements) associated with the patient brain state parameter may be represented, for example, using matrix ATi,j, where i denotes the neurofeedback treatment, j denotes the value requirement of the treatment parameter (e.g., reward requirement), and AT denotes the Administered Treatment, or represented using other suitable multi-dimensional data structures.

At 410, one neurofeedback treatment is selected from the set of neurofeedback treatments for administration to the patient at each iteration. The neurofeedback treatment may be selected randomly from the set of neurofeedback treatments, or the neurofeedback treatment may be ordered and administered sequentially according to the order.

Optionally, neurofeedback treatments are not administered repetitively when performing the adaption and/or selection process. During a subsequent iteration, neurofeedback treatments administered in earlier iterations are excluded from selection.

At 412, the selected neurofeedback treatment is administered to the patient. For example, the patient wears neurofeedback headset 202, and watches images or movies on display (user interface 222). The image (and/or video) and/or sound is modulated according to a comparison of the calculation based on the output of sensor(s) 212 (e.g., EEG sensor) to the value of the requirement target. For example, when the absolute power value of the Alpha frequency EEG signal measured based on output of a selected electrode 212 is within the requirement value range the video is shown normally, and the video is blackened when the power value is outside the requirement range.

At 414, an effectiveness parameter associated with the neurofeedback treatment administered to the patient is calculated. The effectiveness parameter is indicative of the ability of the brain of the patient to produce the desired results in terms of generating desired brain signals. The effectiveness parameter is designed to capture the real-time effects on brain signals achieved by the neurofeedback treatment, which may be analyzed to determine the effectiveness of the neurofeedback treatment.

The effectiveness parameter is calculated based on measured outputs of brain signal(s) outputted by sensor(s) sensing the head of the patient, for example, calculated based on EEG signals outputted by EEG sensors.

Optionally, the effectiveness parameter is calculated over the time duration of each appearance event of a target signal, regardless of whether the reward threshold of the applied neurofeedback treatment is met or not. The effectiveness parameter may be calculated using a function applied to the sensed brain signals, optionally a sum or integral, which may be weighted or an absolute value.

The effectiveness parameter may be calculated as the sum (or integral) of the time duration of each appearance event of a value calculated for a target signal pattern determined based on output of at least one brain signal. The target signal pattern represents the desired goal of the Neurofeedback treatment, e.g., a treatment aimed at strengthening and/or increasing the appearance of Alpha waves at a certain sensor for a certain patient will measure the effectiveness of the treatment based on how strong (e.g., peak energy) and for what duration did the treatment cause Alpha waves to appear at the certain sensor for the certain patient, for example, summing all of the signal amplitudes (e.g., absolute measured level) multiplied by the time duration of the Alpha wave appearance event. The value calculated for the target signal pattern determined based the brain signal may include a power value of each appearance event of a target type of brain activity calculated from electroencephalogram (EEG) signals.

The effectiveness parameter may be calculated based on local maximum values representing peaks of the brain signals, and/or local minimum values representing troughs of the brain signal. Optionally, the effectiveness parameter is calculated based on the time duration of each respective appearance event of the value determined based on a threshold requirement represent one or more local maximum of the target signal pattern, or one or more local minimum of the target signal pattern.

It is noted that a placebo neurofeedback treatment (e.g., as described herein) may be included within the identified set. The placebo neurofeedback treatment may be selected during an iteration, for example, to provide a reference value for evaluation of other neurofeedback treatments. For example, the effectiveness parameter (e.g., as described herein) may be calculated for the placebo neurofeedback treatment, and used to evaluate the efficacy of other real neurofeedback treatments relative to the placebo, for example, serving as a baseline for normalization of the effectiveness parameters calculated for real treatments. For example, effectiveness parameters calculated for real neurofeedback treatments may be evaluated using the effectiveness parameter calculated for the placebo.

The effectiveness parameter may be mathematically represented, for example, as Eff(ATi,j)=SUM (DEi×TDi), where DE denotes the appearance of a desired EEG event that is the target of a desired neurofeedback therapy, TD denotes the time duration of the desired event (e.g., in milliseconds or microseconds), and i denotes the neurofeedback treatment being administered for which the desired EEG events appear in the time interval.

For example in the case of neurofeedback therapies to enhance memory, the goal of all the various ATi,j neurofeedback treatments may be to teach the brain to perform more frequent appearances of stronger Alpha frequencies. The absolute power values of each appearance of a target Alpha brain activity multiplied by its time duration is summed, a concept which may be described as being similar to computing the integral of all appearances of Alpha activity for a given ATi,j neurofeedback treatment.

At 416, blocks 410-414 are iterated to select another neurofeedback and/or another value for the treatment parameters. At each iteration, another combination of a certain neurofeedback treatment and certain treatment parameters may be selected, administered, and measured by calculating the effectiveness parameter. Optionally, at each iteration, each neurofeedback treatment is administered for an approximately equal range of time, for example, about 1 minute, or about 5 minutes, or about 10 minutes, or other values. Alternatively or additionally, the neurofeedback treatment may be administered until a stop condition is met, for example, according to a requirement of the effectiveness parameter, which may be indicative that the treatment is not effective for the patient (the applied treatment may be terminated) or may be indicative that the treatment appears effective for the patient (the applied treatment may continue for a longer time interval).

The iteration may be performed until a stop condition is met, for example, all neurofeedback treatments are administered, all combinations of neurofeedback treatment and treatment parameters are administered, a neurofeedback treatment is found satisfying an effectiveness parameter requirement (e.g., above a threshold or within a range), or a certain amount of neurofeedback treatments of the set have been administered (e.g., a percentage, or absolute number).

At 418, an effective neurofeedback treatment is designated according to the measured effectiveness parameter. The effective neurofeedback treatment may be designated based on the highest calculated effective parameter.

Multiple effective neurofeedback treatments may be designated. The multiple designated treatments may be designated as the set of neurofeedback treatments during another set of iterations to select the best effective neurofeedback treatment from the multiple effective neurofeedback treatments. Alternatively, the multiple effective neurofeedback treatments may be designated for application to the patient, for example, by randomly selecting from the designated treatments, and/or sequentially applying the treatments (e.g., in a round-robin manner).

The effective neurofeedback treatment may be a temporary designation when selected from a sub-set of the set of neurofeedback treatments (i.e., not all combinations and/or treatments have been tested yet), for example, when time does not allow to complete the testing process, or when treatment is scheduled to begin before the testing process is complete, or as part of the testing process. In such a case, the designated effective neurofeedback treatment may be administered to the patient for a range of time (optionally predefined) longer than the range of time of administration of each respective neurofeedback treatment that was tested. The administration of the designated effective neurofeedback treatment may occur, for example, for a factor of about 2, 3, or 5 relative to the testing time (e.g., when the test time is about 5 minutes per treatment, the designated effective neurofeedback treatment may be administered for about 15 minutes).

At 420, wherein the iterating is performed for the subset of neurofeedback treatments, and the temporary designated effective neurofeedback treatment is administered, another set of iterations is performed for the remaining members of the set of neurofeedback treatments that were not members of the iterated subset, by repeating one or more of blocks 410-418. Another effective neurofeedback treatment is designated and administered for another predefined range of time that is longer than the predefined range of time of administration of the previous effective neurofeedback treatment.

The another effective neurofeedback treatment may be designated as the primary neurofeedback treatment, or another iteration cycle may be performed for another subset.

Alternatively, the iteration cycle is repeated using the same members of the set of neurofeedback treatments (e.g., all members) to verify that the same effective neurofeedback treatment is designated both (or a great number of) times. When a neurofeedback treatment is designated (e.g., represented as AT**) that is different than the previously designated neurofeedback treatment (e.g., represented as AT*), the newly designated neurofeedback treatment may be applied for significantly longer time duration than the previously designated effective neurofeedback treatment. For example, 2, 3, or 5 times longer, which may be represented as L>>K, where L and K may be predefined values.

At 422, the evaluation (e.g., of block 404) may be re-administered to obtain another score for the patient after the effective neurofeedback treatment is designated and applied. The scores may be used to monitor the patient receiving the designated neurofeedback treatment. Multiple scores may be obtained during different treatment sessions and/or during different testing sessions. The scores maybe compared to each other, for example, by plotting on a graph and/or using statistical correlation methods to determine whether there are statistically significant changes associated with the scores and the designated treatments. For example, to determine an improvement (or stability) in the MMSE score associated with the designated neurofeedback treatment, why may indicate that the cognitive impairment of the patient has improved or stabilized with the designated neurofeedback treatment.

Optionally, when the analyzing of the scores indicates no improvement in the state of the brain of the patient, or a decrease in the state of the brain of the patient (which is greater than expected), the neurofeedback treatment previously designated as effective may be removed from the testing process, and/or a new neurofeedback treatment may be selected, and/or the neurofeedback treatment may be adjusted (e.g., selection of new treatment parameters) by iterating one or more blocks of the method. For example, when there is no longer any statistically significant change between scores of a memory evaluation comparing before and after significant treatment sessions using the effective neurofeedback treatment (where the goal of the patient is memory improvement), another neurofeedback treatment may be designated by the testing method.

Optionally, the method is repeated occasionally, to evaluate whether the designated effective neurofeedback treatment is still effective for the patient, for example, once a month, once every six months, after every 5 or 10 neurofeedback treatment sessions, or other times or events. The effective neurofeedback treatment may be replaced with another treatment, the treatment parameters may be adjusted, or the effective neurofeedback treatment may be confirmed as still being valid for the patient. In this manner, neurofeedback treatments are adapted to changes to the brain of the patient over time.

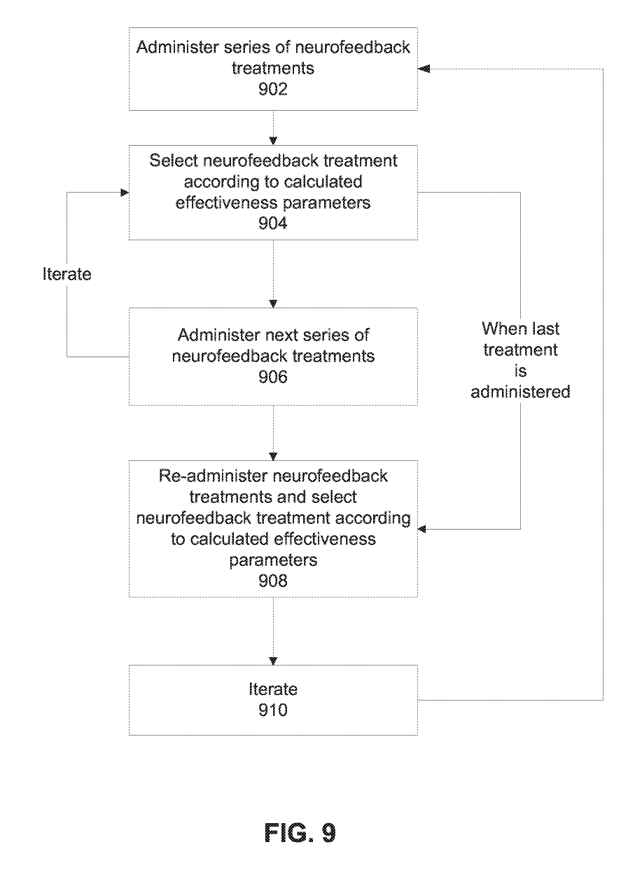

Reference is now made to FIG. 9, which is a flowchart of another exemplary method of selecting effective neurofeedback treatments based on calculation of the effectiveness parameters, in accordance with some embodiments of the present invention. The method described with reference to FIG. 9 may be an implementation based on the method described with reference to FIG. 4. The method described with reference to FIG. 9 utilizes matrix ATi,j, and calculates the effectiveness parameters Eff(ATi,j)=SUM (Dei×TDi), as described with reference to FIG. 4.

At 902, the patient is administered a series of various neurofeedback treatments defined by matrix ATi,j. Each neurofeedback treatment may be administered for a similar time interval T (e.g., a fixed interval period, for example, in minutes, for example, about 1, or 5, or 10 minutes, or other times). For each ATi,j treatment administered, Eff(AT_{i,j}) is calculated, as described herein.

At 904, from the ATi,j neurofeedback treatments administered in block 902, the AT* treatment is selected which has the maximum Eff value (or minimum, depending on the goals of the Neurofeedback therapy). A series of K (e.g. a fixed number) of such AT* treatments is administered to the patient.

At 906, the next series of ATi,j treatments from the matrix is administered. The instruction code according to block 904 is run.

When the last neurofeedback treatment from the ATi,j matrix is administered, the instruction code according to block 904 is run, followed by the instruction code of block 908, without performing the instruction code of block 906.

At 908, the previously administered neurofeedback treatments are re-administered, optionally sequentially (one after the other). The AT** neurofeedback treatment which delivers the maximum (or minimum) Eff value is selected. A series of L number of such AT** treatments is administered to the patient, where L is a fixed number and L>>K.

At 910, the instruction code according to block 902 is re-executed. Referring now back to FIG. 1, at 108, the patient is treated using the designated and/or adapted neurofeedback treatment, as described herein, for example, with reference to FIG. 4. The neurofeedback treatment may include multiple treatment sessions for administration at different times.

Optionally, code stored in program store 238 of computing unit 208 presents a GUI on display (e.g., user interface 224) related to preparation for current treatment session. The GUI may include information, for example, explaining to the user how to conduct the session, for example, how to sit, and how to be comfortable, explain progress towards the recommended total number of treatment sessions (e.g., what has been accomplished and/or what remains to be accomplished), guide the user in choosing preferred content for the treatment (e.g., which video, game, and/or sound track), and guide the user through breathing exercises to enable relaxation.

The user may select to skip screens (e.g., that the user is familiar with), for example, by pressing a forward button.

Optionally, code stored in program store 238 of computing unit 208 presents a GUI on display (e.g., user interface 224) related to guidance for conducting the treatment session, for example, what will happen in the session, what the user should do when the video, game, or audio is playing, and how feedback on performance is evaluated. The guidance may be presented as one or more short movies (e.g., 2-3minutes long). The user may select to skip the guidance videos.

Each neurofeedback session of the neurofeedback treatment may be about 20-30 minutes long. The neurofeedback session may include a movie to watch, a game to play, or a sound track to listen to. The patient is instructed to watch the movie, play the game, or listen to the sound track, while neurofeedback headset 202 measures activity of the brain of the patient and transmits sensed brains signals to computing unit 208 for the analysis. When the brain activity is according to a requirement (e.g., defined by the parameters for the determined effective neurofeedback treatment, as described herein) the movie is played correctly, the game proceeds normally, and/or the sound track is played correctly.

When the brain activity is not according to the requirement, the movie, game, and/or sound track are adapted, for example, the movie begins to blur. Optionally, the greater the value between the current calculated value based on the sensed brain signals and the requirement, the more the movie is blurred, or alternatively, the blurring occurs at the same amount regardless of the value differences between the calculated value and the requirement.

Optionally, when the movie is blurred for at least a predefined period of time (e.g., 30 seconds, 1 minute), an icon is presented overlaid on the blurred movie to help encourage the patient to continue (e.g., focus), for example, a message, a smiley face, or a thumb's up.

Alternatively or additionally, at 110, a placebo version of a neurofeedback treatment is determined for administration to the patient. The placebo neurofeedback treatment may be determined, for example, as part of a clinical trial evaluating a real neurofeedback treatment for efficacy relative to the placebo, for example, a single or double blind randomized clinical trial. In another example, the placebo neurofeedback treatment may be administered to establish a baseline for the patient. The effectiveness parameter may be calculated based on the administered neurofeedback treatment. The placebo effectiveness parameter may be used to normalize or adjust the values of effectiveness parameters calculated for non-placebo neurofeedback treatments, to obtain a more accurate effect of each neurofeedback treatment to help in selecting the effective neurofeedback treatment.

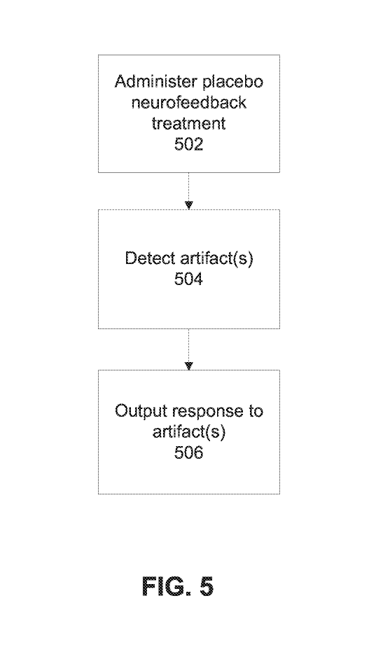

Reference is now made to FIG. 5, which is a flowchart of a method for creating a placebo neurofeedback treatment, in accordance with some embodiments of the present invention. The placebo neurofeedback treatment may be implemented and administered using system 200 described with reference to FIG. 2.

At 502, a non-therapeutic neurofeedback treatment is administered to the patient, optionally using neurofeedback headset 202 and computing unit 208. The non-therapeutic neurofeedback treatment is designed to act as a placebo, by simulating a real neurofeedback treatment without providing therapeutic benefit. The adaptation of video (or other images) and/or audio may be performed randomly, being un-correlated (e.g., according to a correlation requirement representing non-statistically significant correlation) with brain signals (e.g., EEG) of the patient, in comparison to a real neurofeedback treatment in which the adaptation of the video and/or audio signals is in response to measurements based on EEG signals recorded from the patient.

At 504, one or more artifacts are detected. The artifacts may be detected by computing unit 208 and/or neurofeedback headset 202. The artifacts are detected based on an analysis of sensed brain signals (e.g., EEG) outputted by sensors 212 (e.g., EEG electrodes) of neurofeedback headset 202. Alternatively or additionally, the artifacts are detected based on an analysis of outputs of other sensors, for example, motion sensors location within neurofeedback headset 202.

The artifact is based on measurements (e.g., of outputs of sensors 212) in response to patient activity that is unrelated to the administration of the non-therapeutic neurofeedback treatment. The artifact may represents one or more of the following patient activities: a blink of the eye of the patient, movement of sensor 212, the patient touching sensor 212, movement of the head of the patient, chewing movement by the patient, and movement of one or more limbs and/or the body of the patient. When the patient performs one or more of the activities, the patient expects a response, for example, an error message, or an extreme adaptation of the presented video and/or audio. Inventors discovered that since the patient expects a response to the activity, a lack of response by system 200 suggests to the patient that they are receiving the placebo treatment instead of the real neurofeedback treatment. Once the patient realizes they are receiving the placebo, the patient data loses validity. Patients may stop participation, realizing that since they are receiving the placebo their participation is futile. Inventors discovered that providing a response to the activity reduces or prevents the patient from realizing that they are receiving the non-therapeutic placebo neurofeedback treatment.

Optionally, the artifact is detected in EEG signal(s) measured based on output of sensor(s) 212 (i.e., EEG electrodes) sensing the head of the patient. The artifact may be detected as a power spike, optionally a saturation of the outputted signal. Alternatively or additionally, the artifact is detected based on non-EEG signals measured from sensor(s) 212 (e.g., EEG electrodes or other sensors), for example, impedance measurements indicating contact between sensor 212 and the head of the patient, motion sensors, or other sensors. The artifact may be detected as an increase in noise.

At 506, output is generated in response to the detected artifact. Optionally, a simulation (or actual recording) of EEG data measured by the EEG sensors is presented on a display in response to the artifact. The output is designed to correspond to what would otherwise be outputted when a similar activity triggers a similar artifact during a real neurofeedback treatment session. For example, when sensed EEG signals (or simulated EEG signals) are presented on a display, the EEG signals are adapted according to the detected artifact. In another example, the video (or other image) and/or audio (used as part of the real neurofeedback treatment session) is adapted according to the detected artifact during the placebo session (without being adapted in response to other EEG signals).

Referring now back to FIG. 1, at 112, the session of the determined effective neurofeedback treatment is ended, for example, the user completed watching the movie, playing the game, and/or listening to the audio track for the time interval defined by the treatment.

The neurofeedback code may execute code instructions at the end of the session. A close-up screen may be presented on the display (e.g., user interface 224). Information regarding the session may be presented, for example, one or more of: statistical for the current session (e.g., memory score, focus score, determined by an evaluation tool), what has been accomplished to date (e.g., number of treatments, patient progress through the treatments), recommendation to continue to perform for optimal results. The patient may be presented with an option to set a reminder for the next session (e.g., transmit a message to the Smartphone of the patient).

At 114, one or more of blocks 102-112 are iterated. Each iteration may represent a session of the neurofeedback treatment. For example, at the first session, the effective neurofeedback treatment may be selected, as described herein. At subsequent sessions (e.g., every session, or every few sessions), the effective neurofeedback treatment may be adjusted, as described herein. Alternatively, the user may undergo multiple placebo sessions, or alternate between real and placebo sessions, according to the clinical trial protocol.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant neurofeedback treatments will be developed and the scope of the term neurofeedback treatment is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range.

For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In the claims:

1. A computer implemented method for adapting a neurofeedback treatment, comprising:
receiving at least one patient brain state parameter indicative of a current brain state of a patient for application of a neurofeedback treatment;
correlating the at least one patient brain state parameter with a set of neurofeedback treatments from a plurality of neurofeedback treatments stored in a dataset;
performing a plurality of iterations during a neurofeedback session for selection of an effective neurofeedback treatment from members of the set of neurofeedback treatments, each iteration comprising:
selecting one neurofeedback treatment from the set of neurofeedback treatments, wherein in each iteration another neurofeedback treatment is selected;
administering the one neurofeedback treatment to the patient;
calculate an effectiveness parameter associated with the one neurofeedback treatment administered to the patient,
wherein the effectiveness parameter is calculated based on measurements of electrical activity of the brain outputted by at least one sensor sensing the head of the patient,
the effectiveness parameter is calculated over a time duration of each appearance event of a target signal denoting a desired goal of the neurofeedback session; and
designating the effective neurofeedback treatment from the set of neurofeedback treatments according to the measured effectiveness parameter.

2. The computer implemented method of claim 1, wherein the effectiveness parameter is calculated as the sum of the time duration of each respective appearance event of a value calculated for the target signal pattern determined based on output of measurements of electrical activity of the brain.

3. The computer implemented method of claim 2, wherein the time duration of each respective appearance event of the value is determined based on a threshold requirement represent a local maximum of the target signal pattern or a local minimum of the target signal pattern.

4. The computer implemented method of claim 1, wherein the effectiveness parameter is calculated regardless of whether the reward threshold of the administered neurofeedback treatment is met or not.

5. The computer implemented method of claim 2, wherein the value calculated for the target signal pattern determined based on output of measurements of electrical activity of the brain comprises a power value of each appearance event of a target type of brain activity calculated from electroencephalogram (EEG) signals.

6. The computer implemented method of claim 1, further comprising administering the selected effective neurofeedback treatment to the patient for a predefined range of time longer than the range of time of administration of each respective neurofeedback treatment.

7. The computer implemented method of claim 6, wherein the iterating is performed for a subset of neurofeedback treatments, the selected effective neurofeedback treatment is selected and administered, and another iterating is performed for the remaining members of the set of neurofeedback treatments that were not members of the iterated subset.

8. The computer implemented method of claim 6, further comprising:
repeating the iterating and the selecting to select another effective neurofeedback treatment; and
administering the another selected effective neurofeedback treatment to the patient for another predefined range of time longer than the predefined range of time of administration of the previous effective neurofeedback treatment.

9. The computer implemented method of claim 1, further comprising associating each member of the set of neurofeedback treatments with a plurality of treatment parameters each representing a different value for a requirement target, wherein a calculation based on output of at least one sensor measuring electrical activity of the brain of the patient is compared to the value of the requirement target.

10. The computer implemented method of claim 9, wherein at least one of an image and a sound is modulated according to the comparison of the calculation based on the output of the at least one sensor to the value of the requirement target.

11. The computer implemented method of claim 9, wherein selecting comprises selecting one neurofeedback treatment from the set of neurofeedback treatments and an associated set of treatment parameters selected from the plurality of treatment parameters, and measuring the effectiveness parameter according to the associated set of treatment parameters.

12. The computer implemented method of claim 11, wherein iterating comprises iterating the combination of neurofeedback treatments and the plurality of treatment parameters.

13. The computer implemented method of claim 1, wherein during each iterating, each one neurofeedback treatment is administered for an approximately equal range of time.

14. The computer implemented method of claim 1, further comprising: administering an evaluation to obtain a first score for the patient at the current brain state of a patient, administering the evaluation to obtain a second score for the patient after the effective neurofeedback treatment is selected, and comparing the first and second scores.

15. The computer implemented method of claim 14, further comprising removing the effective neurofeedback treatment from use in the iterating when the comparison of the first and second scores is not statistically significant.

16. The computer implemented method of claim 1, wherein the at least one patient brain state parameter is selected from the group consisting of: memory improvement, attention improvement.

17. The computer implemented method of claim 1, wherein when the at least one patient brain state parameter comprises memory improvement, the set of neurofeedback treatments comprise at least one of: absolute power value of the Alpha frequency measured at a selected electrode, relative power of the Alpha frequency compared to the power of the rest of all other frequencies measured for the selected electrode, average power of the measured Alpha frequency over time, and coherence between the phases of the Alpha frequency of several electrodes.

18. The computer implemented method of claim 1, wherein each one neurofeedback treatment is randomly from the set of neurofeedback treatments without repeating selection of a previously selected neurofeedback treatment.

19-26. (canceled)

27. An element for placement of a neurofeedback headset at a predefined position on a head of a patient, comprising:
a first end portion for coupling to an anterior portion of the neurofeedback headset;
an elongated portion extending from the first end portion, the elongated portion having a length such that when the neurofeedback headset is located at the predefined positioned the elongated portion extends parallel to the surface of the frontal bone of the patient until the glabella of the patient;
a pair of arms each extending laterally in opposite directions and inferiorly from the end portion of the elongated portion positioned at the glabella, each respective arm oriented for positioning along at least one of the respective side of the nasal bone and inferiorly to the respective eyebrow and superiorly to the respective eye of the patient.

28. The element of claim 27, wherein the elongated portion is positioned and biased to contact the skin surface of the frontal bone.

29. The element of claim 27, wherein the length of the elongated portion is adjustable to fit patients having different fontal bone surface sizes.

30. The element of claim 27, wherein the end portion of each arm of the pair of arms includes a contact element for contacting the skin of the patient, wherein the contact element is sized and positioned away from the respective eye of the patient.

31. The element of claim 27, wherein the end portion of each arm of the pair of arms is positioned medially to a respective supraorbital notch.

32. The element of claim 27, wherein when the neurofeedback headset comprises a plurality of EEG electrodes that output EEG signals when contacting the head of the patient when the neurofeedback headset is positioned at the predefined position.

33-40. (canceled)

41. The computer implemented method of claim 1, wherein at each iteration, the one neurofeedback treatment is administered for less than about 10 minutes.
